(19) United States
(12) Patent Application Publication
(10) Pub. No.: US 2007/0036758 A1
(43) Pub. Date: Feb. 15, 2007

(54) Mutants of vaccinia virus as oncolytic agents

(76) Inventors: Bertram Jacobs, Tempe, AZ (US); Chandra Mitnik, Tempe, AZ (US); Jeffrey Langland, Chandler, AZ (US)

Correspondence Address: NEEDLE & ROSENBERG, P.C., SUITE 1000, 999 PEACHTREE STREET, ATLANTA, GA, 30309-3915, US

(21) Appl. No.: 10/563,728

(22) PCT Filed: Jul. 8, 2004
(86) PCT No.: PCT/US04/22165

§ 371 Date: Jul. 21, 2006

Related U.S. Application Data
(60) Provisional application No. 60/485,503, filed on Jul. 8, 2003.

(51) Int. Cl. A61K 48/00

(52) U.S. Cl. 424/93.2; 514/44

(57) ABSTRACT

The present invention relates to mutant oncolytic vaccinia viruses and their use for selective destruction of cancer cells. The mutant vaccinia viruses of the invention include those having a reduced ability to inhibit the antiviral dsR-NA dependent protein kinase (PKR) and increased sensitivity to interferon. Such mutants include, for example, vaccinia viruses having mutations in the E3L and/or K3L regions. The invention is based on the discovery that vaccinia viruses having mutations in the E3L region are capable of replication in oncogenic cells resulting in cell lysis. The invention further provides methods for treating proliferative disorders, such as neoplasms, in a host comprising administration of mutant vaccinia virus under conditions which result in substantial lysis of the proliferating cancer cells.

This application claims priority to U.S. Provisional Application No. 60/485,503, filed Jul. 8, 2003.

### FIELD OF THE INVENTION

The present invention relates to mutant oncolytic vaccinia viruses and their use for selective destruction of cancer cells. The mutant vaccinia viruses of the invention include those having an increased sensitivity to interferon. Such mutants include, for example, vaccinia viruses having mutations in the E3L, and/or K3L regions of vaccinia virus (gene notations used are for the Copenhagen strain of vaccinia virus). The invention is based on the discovery that vaccinia viruses having mutations in the E3L region are capable of replication in oncogenic cells resulting in cell lysis. The invention further provides methods for treating proliferative disorders, such as neoplasms, in a host comprising administration of mutant vaccinia virus under conditions which result in substantial lysis of the proliferating cancer cells.

### BACKGROUND OF THE INVENTION

Most current cancer treatments have some selectivity for cells that divide rapidly, such as cancer cells, intestinal cells, and hair follicle cells, but ultimately fail to take advantage of the molecular differences between tumor and normal cells. Oncolytic (“onco” meaning cancer, “lytic” meaning killing) viruses represent a promising new cancer therapy that seeks to exploit the natural properties of viruses to aid in the fight against cancer. Oncolytic viruses are viruses that infect and replicate in cancer cells, destroying the cancer cells and leaving normal cells largely unaffected. Such viruses include reoviruses (Wilcox et al., 2001, J. Natl. Cancer Inst. 93:903-912; Coffey et al., 1998, Science 2:83:1332-133 1; Norman et al., 2002, Human Gene Therapy 13:641-642; Strong et al., 1998, 12:3351-3362), vesicular stomatitis virus (VSV) (Stojdl, 2000 Nature 6:821-825), herpes simplex virus (HSV) (Farasetti et al., Nature Cell Biology 3:745) and human influenza A virus (Bergmann et al., 2001 Cancer Research 64:8188-8193).

The interferon system is a potent anti-viral and anti-tumor system. Interferons work by leading to a signal transduction pathway that leads to induction of antiviral and anti-tumor genes, including PKR and the OAS/RNase L pathway. Interferon has shown some success as an anti-cancer agent. However, numerous cancers have been shown to have mutations which make them non-responsive to interferon. These include mutations in interferon-signaling pathways, mutations in RNase L, and mutations in the ras signaling pathway that lead to induction of an inhibitor of PKR. Thus, an interferon sensitive virus will be able to preferentially replicate in tumor cells that have become non-responsive to interferon, but will replicate poorly or not at all in interferon-responsive non-cancerous normal cells.

The ras protein plays a central role in a variety of cellular processes in vertebrates and invertebrates. Active ras, through a kinase cascade, is responsible for cell differentiation and proliferation in response to normal mitogenic signals. A mutation in the ras gene can cause uncontrolled cell growth, leading to tumor formation. It has been demonstrated that a large number of tumors contain a mutated ras gene that results in a constitutively expressed or always active form of ras, thus proving to be an effective genetic marker of tumor cells and a potential attractive target for therapy.

In addition to these cell growth activities, the ras pathway alters the anti-viral interferon pathway. The interferon system acts as an alarm for the host by warning nearby cells of an impending virus attack. After a cell receives the warning signal of interferon, a biochemical cascade is activated resulting in the induction of hundreds of genes. Among these genes induced by interferon, is the well-studied antiviral dsRNA-dependent protein kinase (PKR). This enzyme becomes activated in the presence of the double-stranded RNA produced during most viral infections. The activated PKR inhibits protein synthesis in order to halt the viral infection. The ras pathway results in an increase in an inhibitor of PKR, which effectively blocks this step in the interferon pathway. This inhibitor has been termed RIKI, which stands for ras-inducible PKR kinase inhibitor. RIKI is believed to be associated with a weak tyrosine or serine/threonine phosphatase activity. Thus, it disables PKR by dephosphorylation, leading to an inactive form of PKR.

Vaccinia virus is highly resistant to treatment of cells with interferon. The E3L and K3L genes are involved in resistance of vaccinia virus to interferon. The E3L gene encodes an inhibitor of the anti-viral and anti-tumor protein PKR and the OAS/RNase L pathway. E3L also inhibits induction of interferon gene expression. K3L encodes a PKR inhibitor. Thus, mutations in one of these genes may make vaccinia virus more sensitive to treatment of cells with interferon, which will allow these viruses to preferentially replicate in interferon non-responsive cancer cells.

### SUMMARY OF THE INVENTION

The present invention relates to mutant oncolytic vaccinia viruses and the use of such viruses for selective destruction of cancer cells. The mutant vaccinia viruses of the invention include those having a reduced ability to inhibit the antiviral dsRNA-dependent protein kinase (PKR) and increased sensitivity to interferon. In some embodiments of the invention, these mutations are in the E3L region or the K3L region.

The invention is based on the discovery that vaccinia viruses having mutations in the E3L region are able to replicate in oncogenic cells resulting in cell lysis. As demonstrated herein, several mutant vaccinia viruses are shown to be oncolytic with specificity for a particular molecular pathway that is commonly dysregulated in a variety of cancers. These vaccinia viruses are dependent on the overexpression of ras (a key molecular characteristic of over 50% of cancers), or of pathways that lead to over-expression of ras, or are dependent on mutations that make cancer cells non-responsive to interferon-treatment. Thus, the present invention provides methods for treating proliferative disorders in a host wherein said method comprises administration of mutant vaccinia virus under conditions which result in substantial lysis of proliferating cancer cells.

Use of vaccinia virus as an oncolytic agent offers several advantages over other oncolytic viruses. First, the viruses can be genetically engineered with ease. Thus, by inserting or deleting genes from vaccinia, the safety and efficacy of the virus can be enhanced. An additional advantage is the wide base of knowledge concerning vaccinia virus infections in humans. Finally, vaccinia virus has been shown to be safe in all but immunocompromised individuals.

By creating various mutants in the vaccinia virus interferon-resistance genes, viruses have been created that are sensitive to interferon. These viruses will preferentially replicate in cancer cells that have lost the ability to respond to interferon, but not in normal interferon-responsive cells. As an example vaccinia virus strains with mutations in the E3L interferon-resistance gene preferentially replicate in ras-transformed mouse cells and in human breast cancer cells but not in normal breast cells.

### BRIEF DESCRIPTION OF THE FIGURES

FIG. 1: Deletion mutants of E3L in vaccinia virus and their PKR inhibitory and ras dependency characteristics.

FIG. 2A-F: Mutant W infections lead to greater cytopathic effect in ras-transformed NIH-3T3cells. NIH-3T3or NIH-3T3 ras-transformed cells were seeded directly onto coverslips and were mock infected or infected with wtVV, VVΔ83N, VVΔ54N, VVΔ7C or VVΔE3L at an MOI of 0.01. At 24, 48, or 72 hpi, cells were fixed, viewed, and photographed using brightfield microscopy.

FIG. 3. Mutant W grows to higher titers in ras-transformed NIH-3T3 cells. NIH-3T3 or ras-transformed NIH-3T3cells were infected at an MOI of 0.01 with wtVV, VVΔ83N, VVΔ54N or VVΔE3L for either 0 or 72 hours.

FIG. 4. A mutant of VV replicates preferentially in select breast cancer cells. Hs 578Bst, Hs 578T, MCF-7, MDA-MD-435s, T-47D, SK-BR-3 or MDA-MB-468 cells were infected at an MOI of 0.01 with wtVV, VVΔ54N, or VVΔE3L for either 0 or 72 hours.

FIG. 5. Ras-transformed NIH-3T3 cells contain an inhibitor of PKR. NIH-3T3 or ras-transformed NIH-3T3 cells were either incubated with IFN to induce production of PKR or were not incubated.

FIG. 6. Select mutants of VV replicates preferentially in SW-480 colon cancer cells. FHC, SW-480, or DLD-1 cells were infected at an MOI of 0.01 with wtVV, VVΔ83N, VVΔ54N, VVΔ26C or VVΔE3L for either 0 or 72 hours.

FIG. 7. A mutant of VV induces oncolytic regression of a breast cancer xenograft. Tumors were induced in SCID/bg female mice by injecting MDA-MD-435s breast cancer cells subcutaneously over both hind flanks. One tumor on each mouse was either mock treated with or treated with VVΔ83N at 1×10⁵ or 1 ×10⁷ pfu, VVΔ54N at 1×10⁵ or 1×10⁷ pfu, or VVΔE3L at 1×10⁵ or 1×10⁷ pfu by intratumoral injection.

FIG. 8. A mutant of W induces oncolytic regression of a breast cancer xenograft. Two tumors were induced in each SCID/bg female mouse by injecting MDA-MD-435s breast cancer cells subcutaneously over each hind flank. One tumor on each mouse was either mock treated with PBS or treated with VVΔ54N at 1×10⁵ or 1×10⁷ pfu by intratumoral injection.

FIG. 9. Treatment of a breast cancer xenograft with select mutants of VV does not cause weight loss. Two tumors were induced in each SCID/bg female mouse by injecting MDA-MD-435s breast cancer cells subcutaneously over each hind flank. One tumor on each mouse was either mock treated with PBS or treated with VVΔ83N, VVΔ54N, or VVΔE3L at 1×10⁵ or 1×10⁷ pfu by intratumoral injection.

FIG. 10. Viral replication by measuring protein synthesis. NIH-3T3 or NIH-3T3 Ha-Ras cells were either mock infected or infected with wtWR, WRΔ83N, WRΔ54N, WRΔ26C, or WRΔE3L.

FIGS. 11A-D. A mutant of VV induces oncolytic regression of a breast cancer xenograft. Two tumors were induced in each SCID/bg female mouse by injecting MDA-MD-435s breast cancer cells resuspended in Matrigel subcutaneously over each hind flank. The right side tumor was treated on each mouse with PBS (mock treatment), UV inactivated virus, WRΔ54N at 1×10⁵ or 1×10⁷ pfu by intratumoral injection. Right side tumors were treated at day 0 and again at day 30 with specified dose. Photographs were taken at 57 days post initial treatment (27 days post second treatment) and are representative of the majority of mice in the particular treatment group.

### DETAILED DESCRIPTION OF THE INVENTION

The present invention relates to mutant oncolytic vaccinia viruses and the use of such viruses for selective destruction of cancer cells. Mutant vaccinia viruses of the invention have an inactivating mutation in an interferon resistance gene. Thus, mutant vaccinia viruses of the invention comprise mutant vaccinia viruses with a reduced ability to inhibit the antiviral dsRNAdependent protein kinase (PKR) and increased sensitivity to interferon. In some embodiments of the invention, these mutations are selected from the group consisting of a deletion mutation (a whole gene or function-critical portion thereof is deleted), a substitution mutation (a whole gene or function-critical portion thereof is replaced by other nucleotides (e.g. another gene)), and missense mutations (a frame-shift or other mutation that alters the encoded amino acid sequence). In particular, the present invention provides recombinant vaccinia viruses for which the region encoding the E3L and/or K3L gene products have been inactivated. Such inactivation may result from partial or complete deletion of the regions or, alternatively, substitution of nucleotides within the regions that result in full or partial inactivation of the gene product.

The invention is based on the discovery that such mutant viruses are unable to inhibit PKR thus rendering the viruses dependent on the PKR inhibitory activity found in ras transformed cells or on the non-responsiveness of many transformed cells to interferon.

The E3L gene product of the vaccinia virus is a 190 amino acid polypeptide. The E3L gene codes for several functions including a dsRNA-binding protein, a Z-DNA-binding protein, and dimerization. Amino acids II 8-190 have been implicated in dsRNA binding, as disclosed by Chang and Jacobs (1993, Virology 194:537-547). Amino acid numbering as used herein is adopted from Goebel et al., 1990, Virology 179:247-66, 577-63.

According to the invention “deletion of the E3L gene” and its grammatical equivalents refer to a vaccinia virus wherein a nucleic acid encoding all 190 amino acids or a subset of the 190 amino acids of E3L are not present. According to the invention, if the vaccinia virus having a deletion in the E3L gene has a residual nucleic acid encoding a subset of the 190 amino acids of E3L, said residual nucleic acid is incapable of producing a fully functional gene product or the gene product is incapable of binding dsRNA. The ability of the E3L gene product to bind to dsRNA can be determined by binding assays known in the art and disclosed, for example, by Chang et al., 1993, Virology 194:537.

Deletion of the E3L gene from vaccinia virus results in a virus that is interferon sensitive, but also is highly debilitated for replication in many cells in culture (Jacobs and Langland, 1996, Virology 219(2):339-349). However, as demonstrated herein, such viruses are capable of replication in ras-transformed cells thereby providing a method for targeted cell lysis of ras-transformed cells.

The recombinant vaccinia virus of the present invention may be constructed by methods known in the art, and preferably by homologous recombination. Standard homologous recombination techniques utilize transfection with DNA fragments or plasmids containing sequences homologous to viral DNA, and infection with wild-type or recombinant vaccinia virus, to achieve recombination in infected cells. Conventional marker rescue techniques may be used to identify recombinant vaccinia virus. Representative methods for production of recombinant vaccinia virus by homologous recombination are disclosed by Piecini et al., 1987, Methods in Enzymology 153:545.

For example, the recombinant vaccinia virus of a preferred embodiment of the present invention may be constructed by infecting host cells with vaccinia virus from which the E3L gene has been deleted. The vaccinia virus used for preparing the recombinant vaccinia virus of the invention may be a naturally occurring or engineered strain. Strains useful as human and veterinary vaccines are particularly preferred and are well-known and commercially available. Such strains include Wyeth, Lister, WR, and engineered deletion mutants of Copenhagen such as those disclosed in U.S. Pat. No. 5,762,938. Recombination plasmids may be made by standard methods known in the art. The nucleic acid sequences of the vaccinia virus E3L gene and the left and right flanking arms are well-known in the art, and may be found for example, in Earl et al., 1993, in Genetic Maps: locus maps of complex genomes, O'Brien, ed., Cold Spring Harbor Laboratory Press, 1. 1 5 7 and Goebel et al., 1990, supra. The amino acid numbering used herein is adopted from Goebel et al., 1990, supra. The vaccinia virus used for recombination may further comprise other deletions, inactivations, or exogenous DNA.

The present invention further provides compositions for use in targeted cell lysis wherein said compositions comprise a recombinant vaccinia virus, or viral vector, and a carrier. The term carrier as used herein includes any and all solvents, diluents, dispersion media, antibacterial and antifungal agents, microcapsules, liposomes, cationic lipid carriers, isotonic and absorption delaying agents, and the like. Suitable carriers are known to those of skill in the art. The compositions of the invention can be prepared in liquid forms, lyophilized forms or aerosolized forms. Other optional components, e.g., stabilizers, buffers, preservatives, flavorings, excipients and the like, can be added.

Also included in the invention is a method of treating a host with cancer, including but not limited to mammals such as a humans, with the novel compositions of the invention under conditions which result in substantial lysis of the proliferating cancer cells. In the method of the invention, the recombinant vaccinia viruses of the invention are administered to ras-mediated, or interferon non-responsive transformed cells in the host. The compositions, including one or more of the recombinant vaccinia viruses described herein, are administered using routes typically used for such administration, i.e., intravenously, intravascularly, injection at site of tumor, in a suitable dose. The dosage regimen involved in the method of treating, including the timing, number and amounts of treatments, will be determined considering various hosts factors, e.g., the age of the patients, time of administration and type and severity of the cancer.

### EXAMPLES

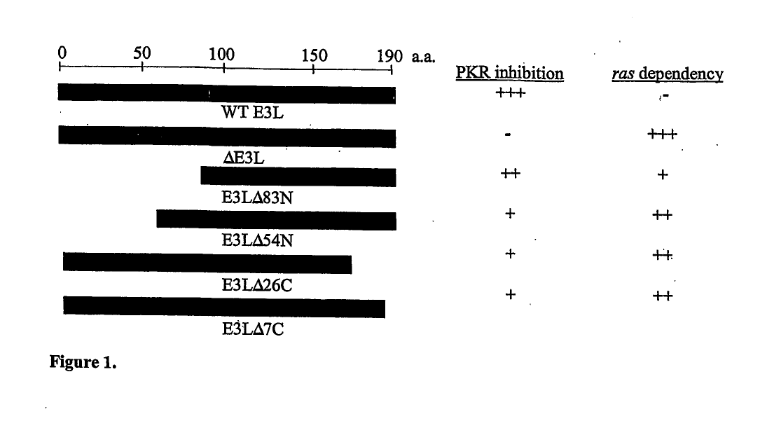

FIG. 1 depicts deletion mutants of E3L in vaccinia virus and their PKR inhibitory and ras dependency characteristics.

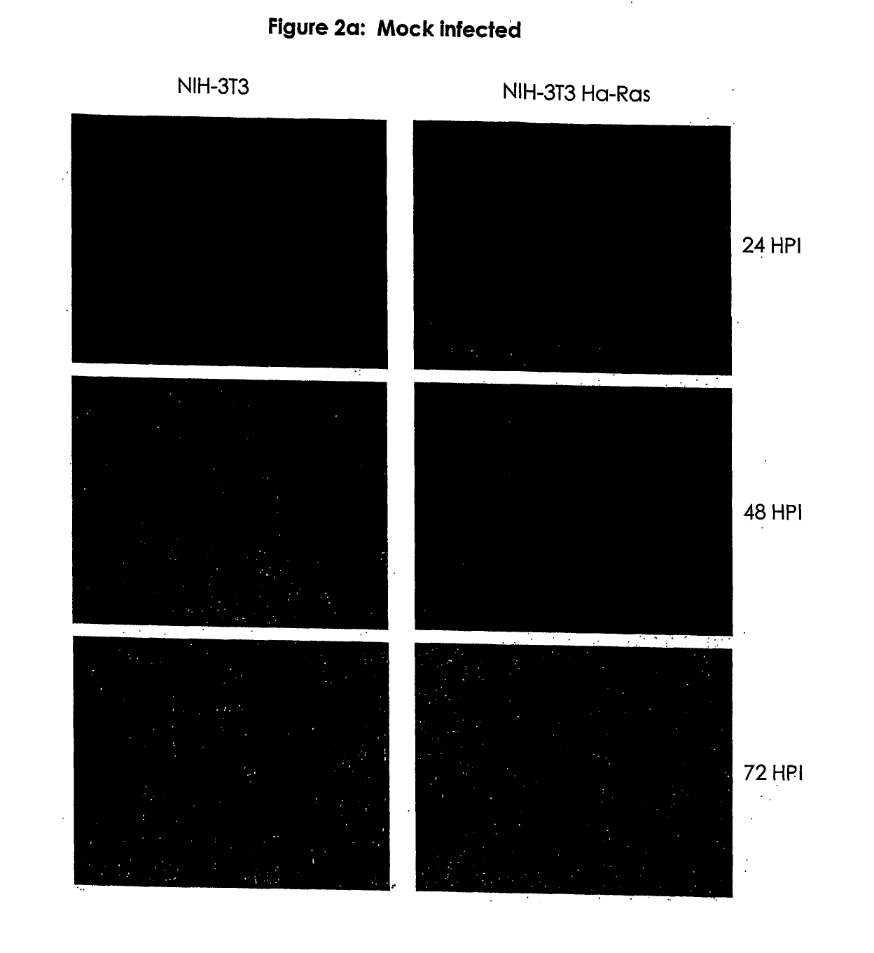

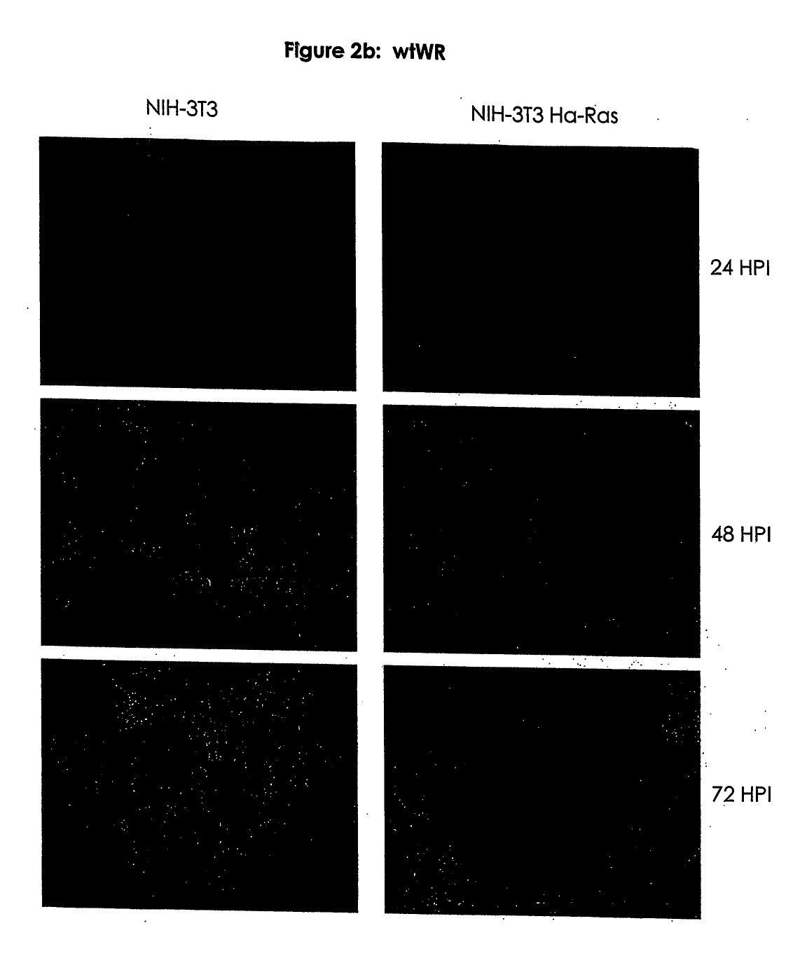

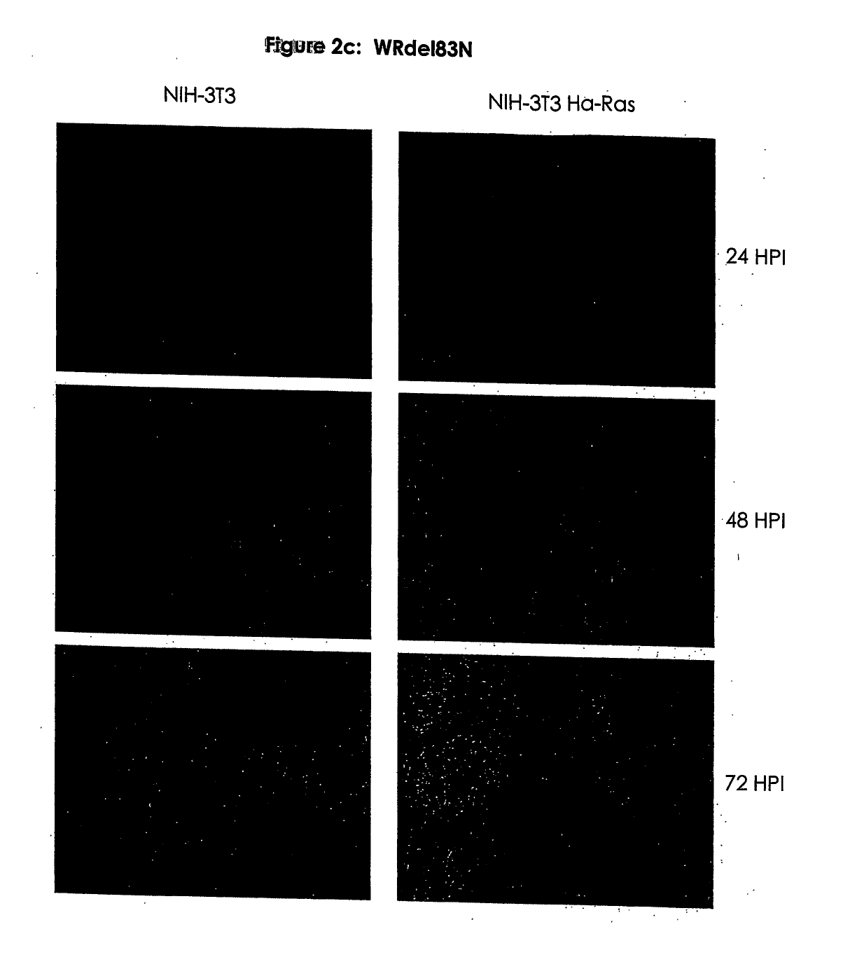

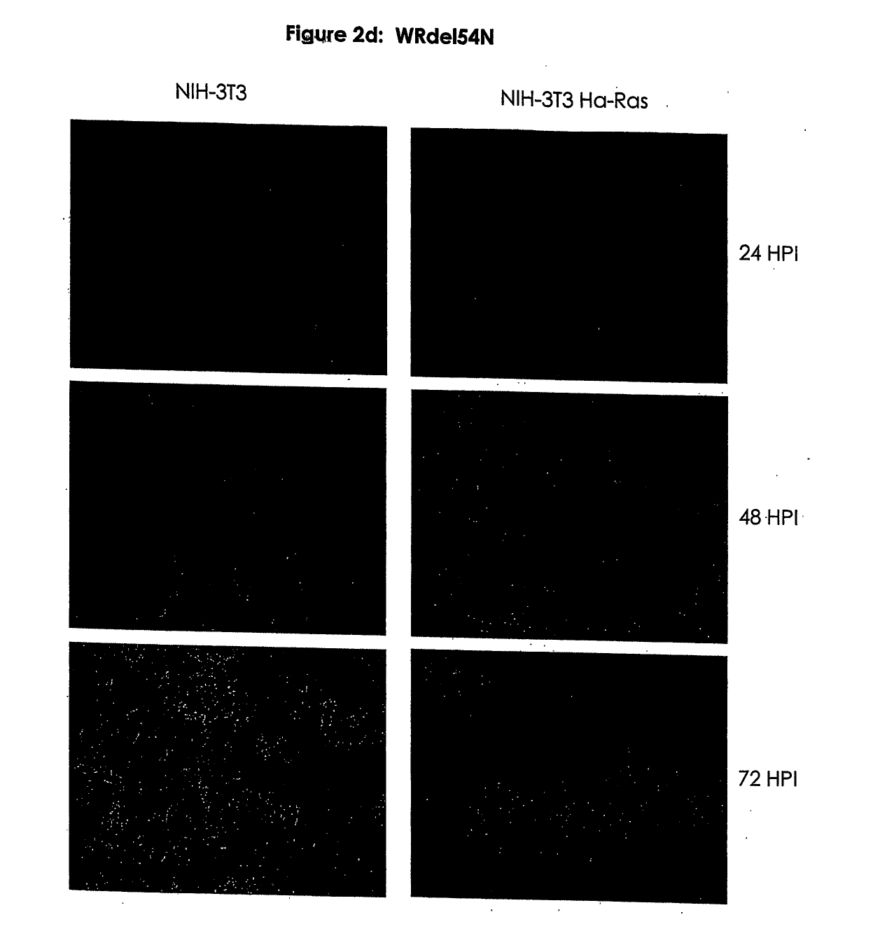

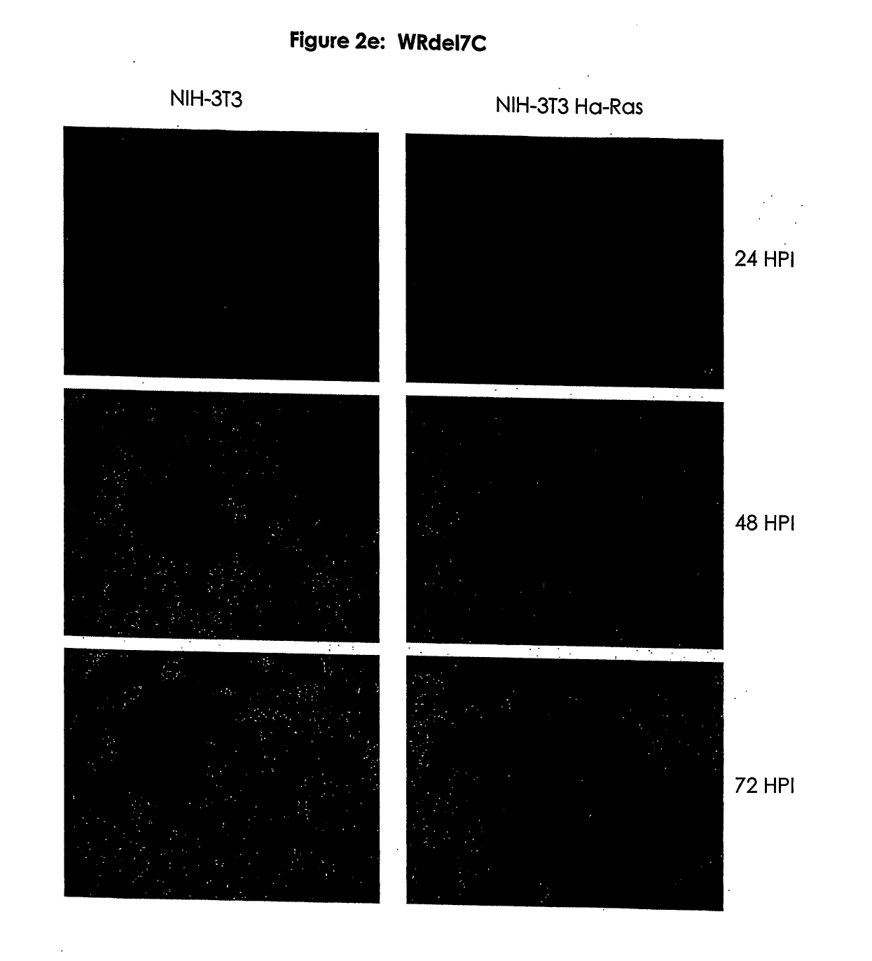

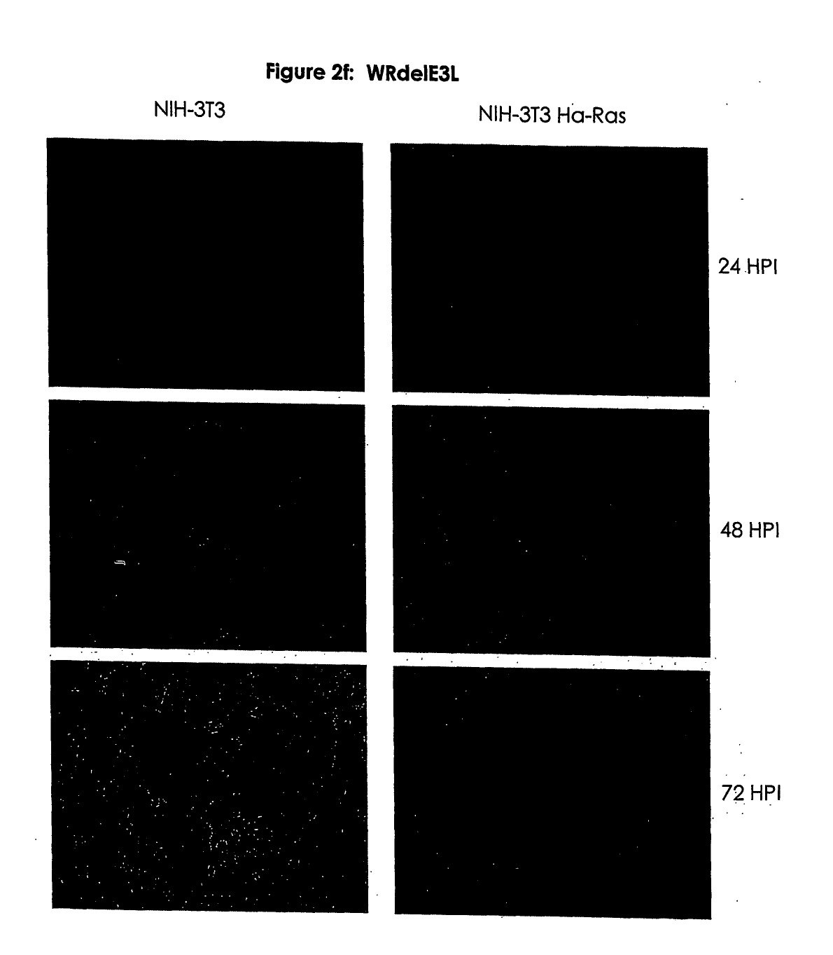

As illustrated in FIGS. 2A-F, mutant infections lead to greater cytopathic effect in ras-transformed NIH-3T3 cells. Here, NIH-3T3 or NIH-3T3 ras-transformed cells were seeded directly onto coverslips and were mock infected or infected with wtVV, VVΔ83N, VVΔ54N, VV(7C or VVΔE3L at an MOI of 0.01. At 24, 48, or 72 hpi, cells were fixed, viewed, and photographed using brightfield microscopy. NIH-3T3 or NIH-3T3 overexpressing the ras protein were either mock infected or infected with the above identified vaccinia virus constructs at an MOI (multiplicity of infection) of 0.01. Cytopathic effect is a description of any adverse properties of cells following infection. Photographs were taken at 24, 48 and 72 hours post infection to record cytopathic effect. In FIG. 2a, all cells were mock infected and appear normal and healthy through 72 hours post infection. In FIG. 2b, cells were infected with wt WR virus, which is not ras-dependent. Cytopathic effect was noted in both the NIH-3T3 and NIH-3T3 Ha-Ras beginning at 48 hours post infection and continuing to 72 hours post infection. In FIG. 2B, cells were infected with wt WR virus, which was not ras-dependent. Cytopathic effect was noted in both NIH-3T3 and NIH-3T3 Ha-Ras beginning at 48 hours post infection and continuing to 72 hours post infection. Slight cytopathic effect was noted in FIG. 2e, when cells were infected with WRΔ7C, indicating that this virus is less ras-dependent than the other mutant viruses. Cytopathic effect was not evident in FIGS. 2C, 2D and 2F in the NIH-3T3 cells, indicating that these virus constructs are ras-dependent.

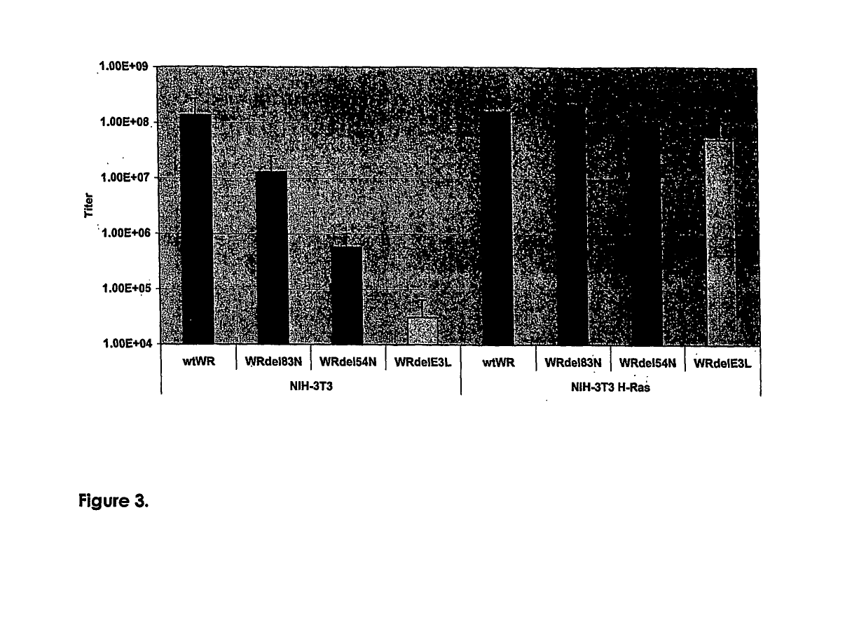

To illustrate that mutant WR grows to higher titers in ras transformed NIH-3T3 cells, NIH-3T3 or NIH-3T3 Ha-Ras cells were infected with wtWR, WRΔ83N, WRΔ54N, and WRΔE3L at an MOI of 0.01. Viral replication was measured by determining how many infectious virus particles were present after 72 hours. The number of infectious virus particles is expressed as titer and is on the y-axis, while the various vaccinia constructs are depicted on the x-axis. WtWR grew to high titers in both cell lines. Titers dropped in the NIH-3T3 cells, but remained high in the NIH-3T3 Ha-Ras cells for all of the vaccinia constructs. FIG. 3 represents viral replication over a 72-hour period. NIH-3T3 or ras-transformed NIH-3T3 cells were infected at an MOI of 0.01 with wtVV, VVΔ83N, VVΔ54N or VVΔE3L for either 0 or 72 hours. After harvesting, viral titers were determined via plaque assay and 0-hour titers were subtracted from 72-hour titers to distinguish viral replication from virus input. This assay was repeated twice and the averages were graphed. Error bars equals standard error.

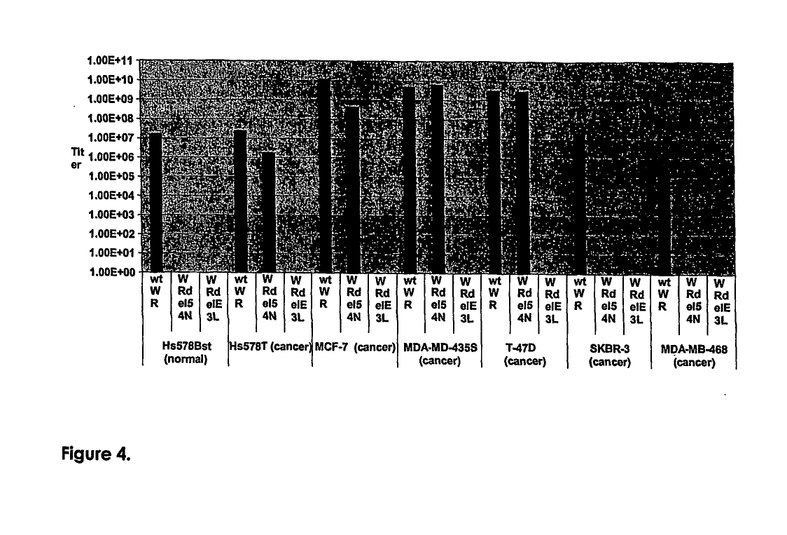

Experiments were completed to illustrate the preferential replication of a mutant of VV in select breast cancer cells. The results are shown in FIG. 4. Hs 578Bst, Hs 578T, MCF-7, MDA-MD-435s, T-47D, SK-BR-3 or MDA-MB-468 cells were infected at an MOI of 0.01 with wtVV, VVΔ54N, or VVΔE3L for either 0 or 72 hours. After harvesting, viral titers were determined via plaque assay and 0-hour titers were subtracted from 72-hour titers to distinguish viral replication from virus input. This figure represents viral replication over a 72-hour period. Either normal breast cells or cancerous breast cells were infected with wtWR, WRde154N, and WRΔE3L at an MOI of 0.01. Viral replication was measured by determining how many infectious virus particles were present after 72 hours. The number of infectious virus particles is expressed as titer and is on the y-axis, while the various vaccinia constructs are depicted on the x-axis. WtWR grew to high titers in all cell lines. WRΔE3L failed to grow in any cell line. WRΔ54N did not grow in the normal breast cells, or in two of the cancer cell lines. However, WRΔ54N grew to high titers in four out of six breast cancer cell lines.

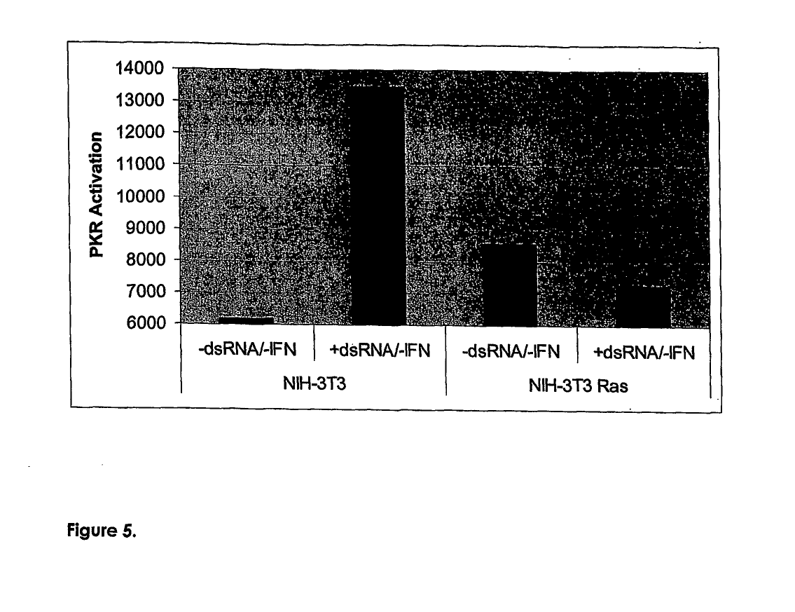

FIG. 5 demonstrates that ras-transformed NIH-3T3 cells contain an inhibitor of PKR. NIH-3T3 or ras-transformed NIH-3T3 cells were either incubated with IFN to induce production of PKR or were not incubated. The cells were harvested and were subjected to an in vitro kinase assay. Cell lysates were incubated with or without dsRNA to activate PKR and radioactively labeled substrate to detect the phosphorylation event which represents PKR activation. The lysates were purified and loaded onto a SDS-polyacrylamide gel. Autoradiography detected any radioactive PKR. The intensity of each PKR band was measured using the computer software ImageQuant and the relative intensities were graphed.

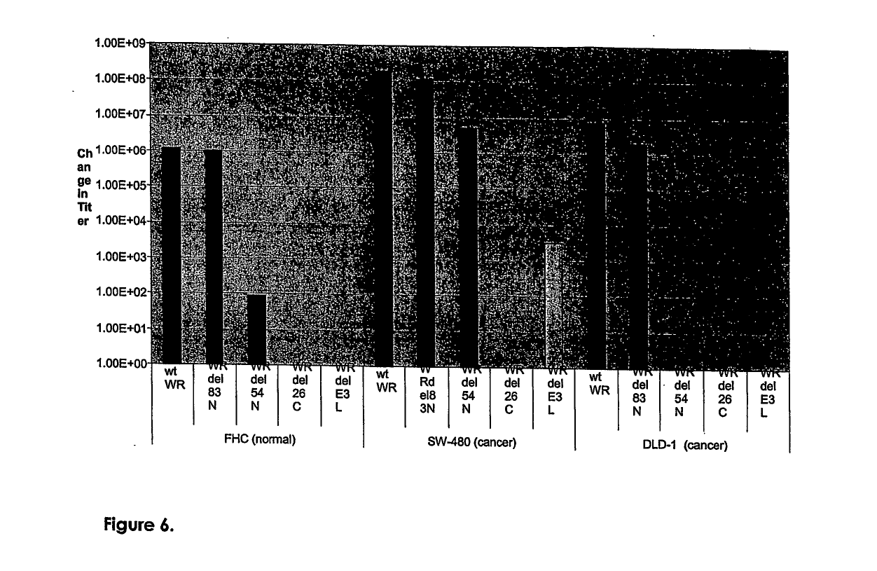

As shown in FIG. 6, select mutants of VV replicate preferentially in SW-480 colon cancer cells. FHC, SW-480, or DLD-1 cells were infected at an MOI of 0.01 with wtVV, VVΔ83N, VVΔ54N, VVΔ26C or VVΔE3L for either 0 or 72 hours. After harvesting, viral titers were determined via plaque assay and 0-hour titers were subtracted from 72-hour titers to distinguish viral replication from virus input.

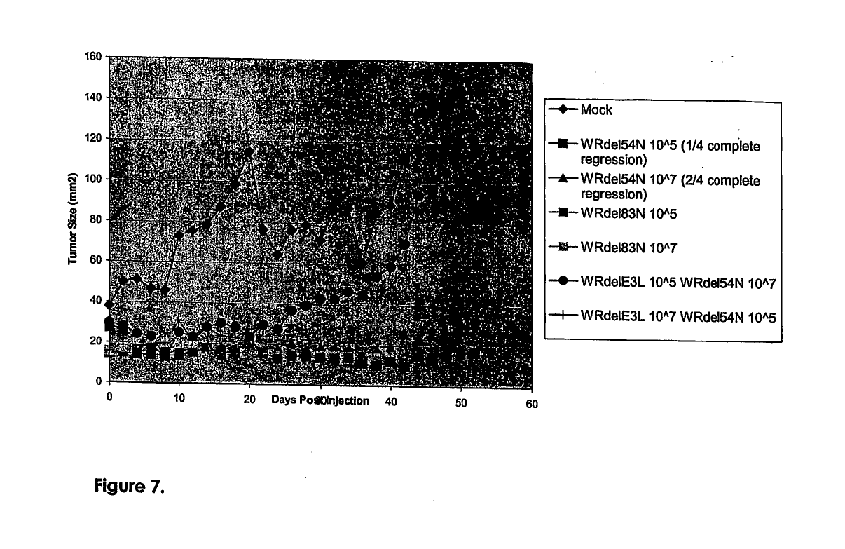

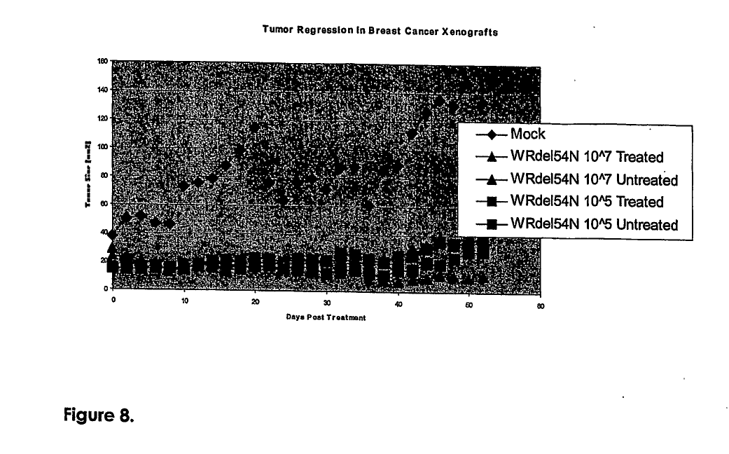

Further, as illustrated in FIGS. 7 and 8, a mutant of VV induces oncolytic regression of a breast cancer xenograft. As shown in FIG. 8, tumors were induced in SCID/bg female mice by injecting MDA-MD-435s breast cancer cells subcutaneously over both hind flanks. One tumor on each mouse was either mock treated with or treated with VVΔ83N at 1×10⁵ or 1×10⁷ pfu, VVΔ54N at 1×10⁵ or 1×10⁷ pfu, or VVΔE3L at 1×10⁵ or 1×10⁷ pfu by intratumoral injection. Tumors were measured every other day for the duration of the experiment. This graph represents tumor that received a treatment of virus or PBS. FIG. 7 depicts two tumors induced in each SCID/bg female mouse by injecting MDA-MD-435s breast cancer cells subcutaneously over each hind flank. One tumor on each mouse was either mock treated with PBS or treated with VVΔ54N at 1×10⁵ or 1×10⁷ pfu by intratumoral injection. Tumors were measured every other day for the duration of the experiment. Each treatment group consisted of four mice. One mouse in mock treatment group was removed from the study at day 22 due to significant tumor burden. At the end of the study, one tumor in the VVΔ54N 1×10⁵ pfu treatment group completely regressed, and three tumors in the VVΔ54N 1×10⁷ pfu treatment group completely regressed.

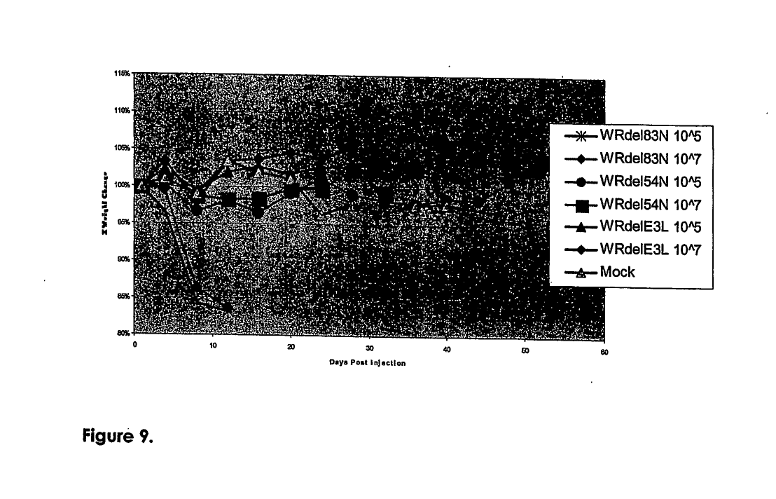

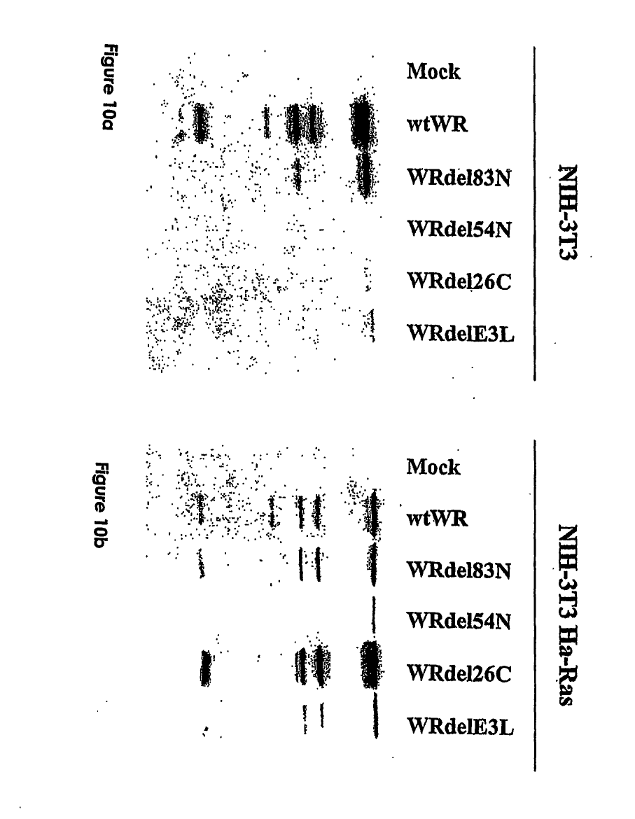

As shown in FIG. 9, treatment of a breast cancer xenograft with select mutants of VV does not cause weight loss. Two tumors were induced in each SCID/bg female mouse by injecting MDA-MD435s breast cancer cells subcutaneously over each hind flank. One tumor on each mouse was either mock treated with PBS or treated with VVΔ83N, VVΔ54N, or VVΔE3L at 1×10⁵ or 1×10⁷ pfu by intratumoral injection. Each treatment group consisted of four mice. Weights of mice were monitored for the duration of the experiment and plotted as a percentage of the initial weight. Treatment with VVΔ83N caused morbidity in this mouse model at 12 days post treatment. The remaining treatment regimens resulted in weight averages higher than that of mock treated animals, indicating safety of treatment.

FIG. 10 depicts viral replication by measuring protein synthesis. NIH-3T3 or NIH-3T3 Ha-Ras cells were either mock infected or infected with wtWR, WRΔ83N, WRΔ54N, WRΔ26C, or WRΔE3L. At 72hours post infection, the cells were harvested and their proteins loaded onto this gel. This gel was then probed with antibodies against vaccinia virus in order to detect vaccinia virus proteins. Vaccinia virus proteins were not detected in either mock infection. Vaccinia virus proteins were detected in wtWR and less in WRΔ83N infected NIH-3T3 cells. Viral protein synthesis was not detected in WRΔ54N, WRΔ26C, or WRΔE3L infected NIH3T3 cells. Viral protein synthesis was detected in all infected NIH-3T3 Ha-Ras cells, with lower levels noted in WRΔ54N infected cells.

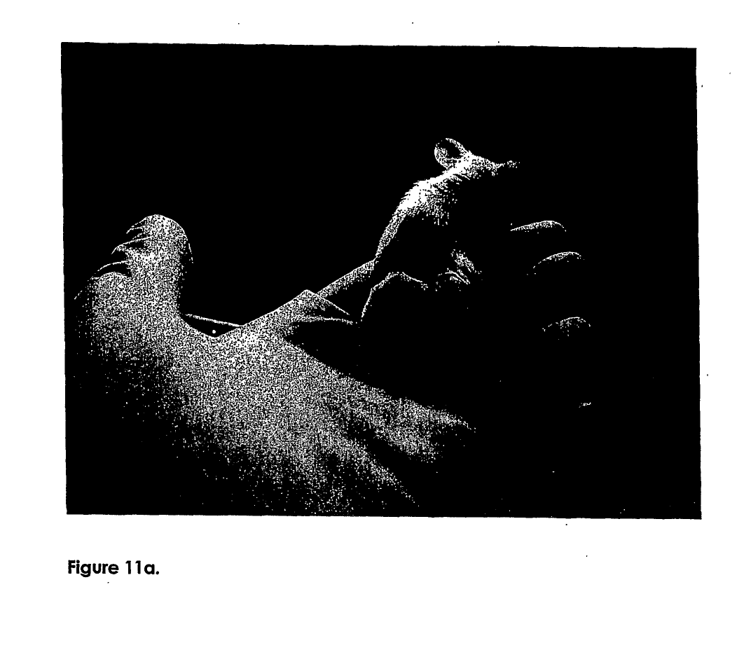

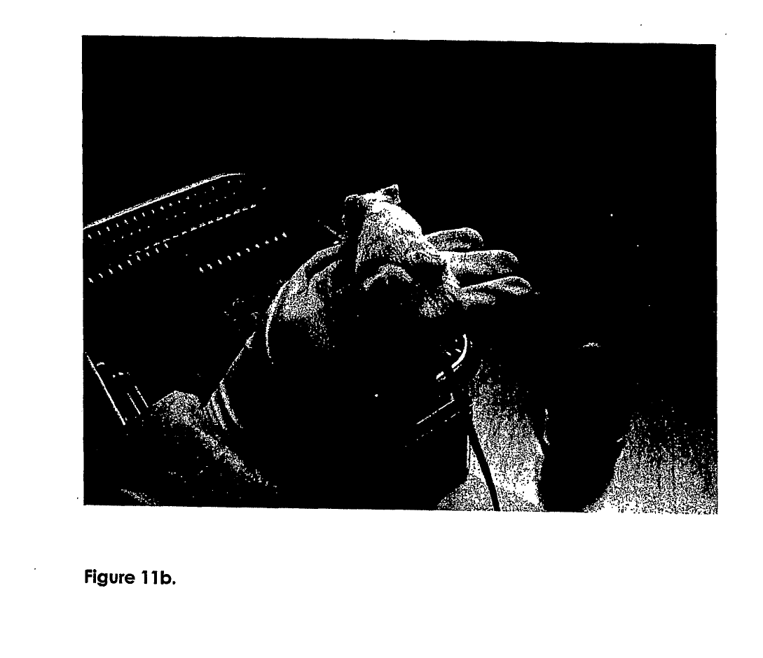

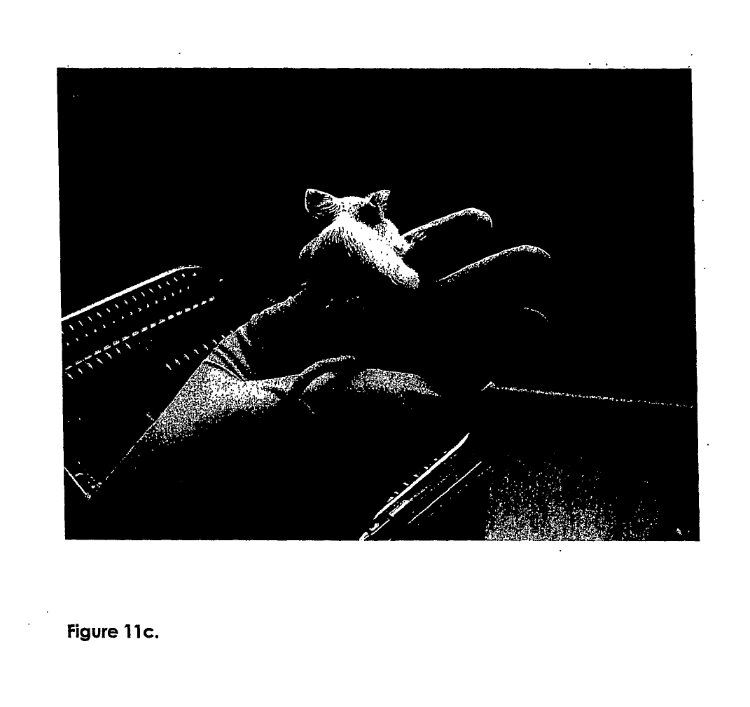

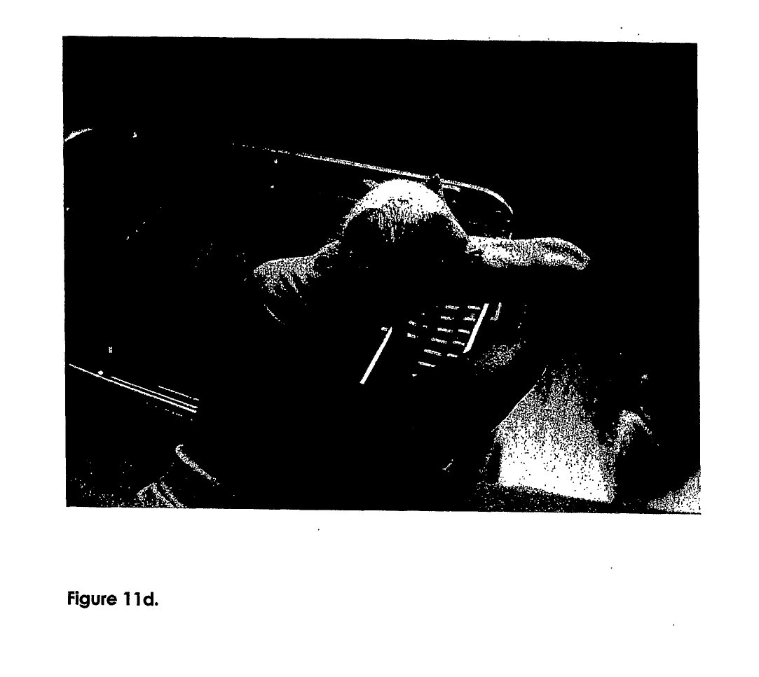

FIGS. 11A-D illustrate that a mutant of VV induces oncolytic regression of a breast cancer xenograft. Two tumors were induced in each SCID/bg female mouse by injecting MDA-MD-435s breast cancer cells resuspended in Matrigel subcutaneously over each hind flank. The right side tumor was treated on each mouse with PBS (mock treatment), UV inactivated virus, WRΔ54N at 1×10⁵ or at 1×10⁷ pfu by intratumoral injection. Right side tumors were treated at day 0 and again at day 30 with specified dose. Photographs were taken at 57 days post initial treatment (27 days post second treatment) and are representative of the majority of mice in the particular treatment group. In FIGS. 11A and 11B, neither tumor responded to mock treatment or to treatment with UV inactivated virus which resulted in tumor growth on both left and right side. In FIG. 11C, the right side tumor that was treated with WRΔ54N at 1×10⁵ responded by regressing while the left side tumor did not respond to treatment. In FIG. 11D, the right side tumor was treated with WRΔ54N at 1×10⁷ and both tumors responded to treatment by regressing. At the end of the experiment, animals were necropsied and the tumors harvested. The tumors directly treated with WRΔ54N at either 1×10⁵ or 1×10⁷ fully regressed. Any residual mass was found to be composed of the Matrigel used to resuspend the breast cancer cells in the initial xenograft.

All sequences, patents, patent applications or other documents cited anywhere in this specification are herein incorporated in their entirety by reference to the same extent as if each individual sequence, publication, patent, patent application or other document was specifically and individually indicated to be incorporated by reference.

We claim:

1. A method of inducing lysis of proliferating cancer cells comprising contacting said cells with a vaccinia virus having an inactivating mutation in an interferon resistance gene.

2. The method of claim 1, wherein the cancer cells are ras-transformed cells.

3. The method of claim 1, wherein the cancer cells are breast cancer cells or prostate cancer cells.

4. The method of claim 1, wherein the inactivating mutation is in a gene selected from the group consisting of E3L, K3L, or a combination thereof.

5. The method of claim 4, wherein the inactivating mutation is selected from the group consisting of a deletion mutation, a substitution mutation, and a missense mutation.

6. The method of claim 4, wherein the inactivating mutation is in the E3L gene.

7. The method of claim 6, wherein the mutation is a deletion of the whole E3L gene.

8. The method of claim 1, wherein the mutant vaccinia virus has a reduced ability to inhibit PKR and increased sensitivity to interferon.

9. The method of claim 1, wherein said contacting comprises administering a therapeutic amount of the vaccinia virus to a mammal comprising proliferating cancer cells under conditions that permit contact between the vaccinia virus and the proliferating cancer cells.

10. The method of claim 9, wherein the administering is selected from the group consisting of intratumoral injection, intravenous injection, and intravascular injection.

11. A therapeutic composition for use in targeted cell lysis of a proliferating cancer cell comprising a vaccinia virus having an inactivating mutation in an interferon resistance gene and a carrier.

12. The therapeutic composition of claim 11, wherein the target cell is a breast cancer cell or prostate cancer cell.

13. The composition of claim 12, wherein the inactivating mutation is in a gene selected from the group consisting of E3L, K3L, or a combination thereof.

14. The composition of claim 13, wherein the inactivating mutation is selected from the group consisting of a deletion mutation, a substitution mutation, and a missense mutation.

15. The composition of claim 13, wherein the inactivating mutation is in the E3L gene.

16. The composition of claim 15, wherein the mutation is a deletion of the whole E3L gene.
